(19) United States
(12) Patent Application Publication
(10) Pub. No.: US 2011/0136748 A1
(43) Pub. Date: Jun. 9, 2011

(54) NOVEL GLUCOPYRANOSE DERIVATIVES, PREPARATION THEREOF, AND BIOLOGICAL USES THEREOF

(76) Inventor: Robert Vachy, Versailles (FR)

(21) Appl. No.: 12/737,748

(22) PCT Filed: Jul. 20, 2009
(86) PCT No.: PCT/IB2009/053131

§ 371 Date: Feb. 11, 2011

(30) Foreign Application Priority Data
Aug. 12, 2008 (FR) 08/04572

(51) Int. Cl. A61K 31/7028; C07H 15/06; C07H 1/00; A61P 31/14; A61P 31/16; A61P 31/18

(52) U.S. Cl. 514/25; 536/18.2

(57) ABSTRACT

The invention relates to a novel ester, the 3,5-di-tertiobutyl-4-hydroxybenzoate of 3,4,5-trihydroxy-6-methoxytetrahydropyran-2-yl methyl defined by the following formula (I). The invention also relates to the method for preparing the compound of formula (I) and to the use of said compound (I) to prepare a drug for treating and/or preventing enveloped virus infections.

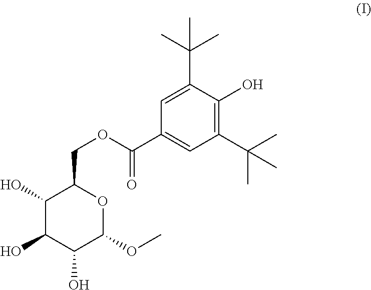

The present invention relates to novel glucopyranose derivatives, in particular a novel ester, namely “3,4,5-trihydroxy-6-methoxytetrahydropyran-2-ylmethyl 3,5-di-tert-butyl-4-hydroxybenzoate”, and to the derivatives of said ester.

The invention also relates to the process for the preparation thereof and to the biological uses of said compound and of derivatives thereof, in particular for treating viral and retroviral infections.

The novel derivative of the invention has a structural formula close to the D-glucopyranose sugar compound previously described in FR 2 887 249 in the name of the applicant, and the use of which is found to be particularly advantageous against infections with enveloped viruses, and in particular the herpesvirus, the AIDS virus, the flu virus, the hepatitis B virus and the hepatitis C virus.

The studies pursued in this field have led the inventor to the novel ester which is the subject of the invention and to derivatives thereof which, unexpectedly, make it possible to further improve the results obtained with the D-glucopyranose sugar compound, for instance greater solubility, greater stability, a perfectly reproducible synthesis, and a greater biological activity at lower concentrations than those of the prior art.

The purpose of the invention is therefore to provide novel active compounds against enveloped viruses.

The invention is also directed toward the use of said compounds for producing antiviral pharmaceutical compositions of great interest.

A subject of the present invention is more particularly the compound 3,4,5-trihydroxy-6-methoxytetrahydropyran-2-ylmethyl 3,5-di-tert-butyl-4-hydroxybenzoate defined by formula (I) below:

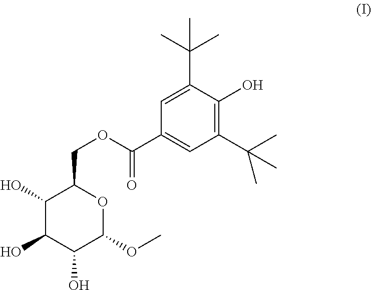

The compound of the invention can also be in the form of a derivative, such as, for example, in the form of a salt or of an acid of said ester (I).

The compound (I) of the invention and also said derivative(s) may be provided in a composition comprising at least one pharmaceutically acceptable excipient.

A subject of the invention is also a composition comprising at least one compound as defined above, in combination with a biologically active compound.

By way of example of biologically active compounds, mention may be made of:
compounds of plant origin,
3,5-di-tert-butyl-4-hydroxybenzoic acid,
octaoxyethylene glycol 3,5-di-tert-butyl-4-hydroxy-benzoate,
nonwoven derivatives of synthetic origin,
derivatives close to cyclopentanophenanthrene,
antiviral and antiretroviral virustatic agents.

A subject of the invention is also a pharmaceutical composition containing a therapeutically effective amount of at least one compound or of at least one composition as defined above.

The amount of compound or of composition may vary according to the applications envisioned, and the age and weight of the patient.

The pharmaceutical composition of the invention may be in a form suitable for oral, injectable or parenteral administration.

By way of example, mention may be made of tablets, sugar-coated tablets, oral or injectable solutions or suspensions, emulsions, suppositories, etc.

Thus, the present invention also covers the use of a compound or of a composition as defined above, for preparing a medicament for treating and/or preventing enveloped virus infections.

A subject of the invention is also a process for preparing a novel ester, 3,4,5-trihydroxy-6-methoxy-tetrahydropyran-2-ylmethyl 3,5-di-tert-butyl-4-hydroxy-benzoate, of formula (I).

This process comprises the prior steps of formation of, on the one hand, 3,5-di-tert-butyl-4-hydroxybenzoic acid chloride of formula (III) and of, on the other hand, [6-methoxy-3,4,5-tris(4-methoxybenzyloxy)tetra-hydropyran-2-yl]methanol of formula (V), and then a coupling reaction between the resulting two compounds (III) and (IV) so as to obtain the protected ester (II), which, after deprotection, gives the glucopyranose compound (I) of the invention.

Thus, the process for preparing the compound of formula (I) of the invention is more particularly characterized in that it comprises reacting 3,5-di-tert-butyl-4-hydroxybenzoyl chloride of formula (III):

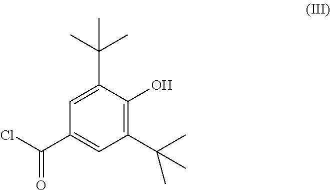

with [6-methoxy-3,4,5-tris(4-methoxybenzyloxy)tetra-hydropyran-2-yl]methanol of formula (V):

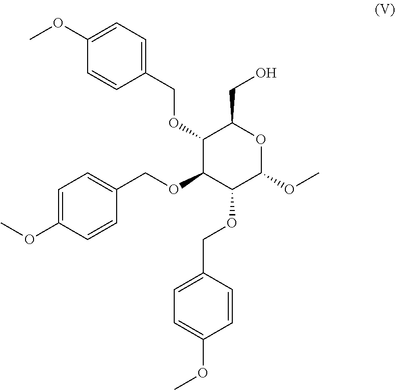

so as to obtain the esterified compound “3,4,5-tris(4-methoxybenzyloxy)-6-methoxytetrahydro-pyran-2-yl]methyl 3,5-di-tert-butyl-4-hydroxybenzoate” of formula (II):

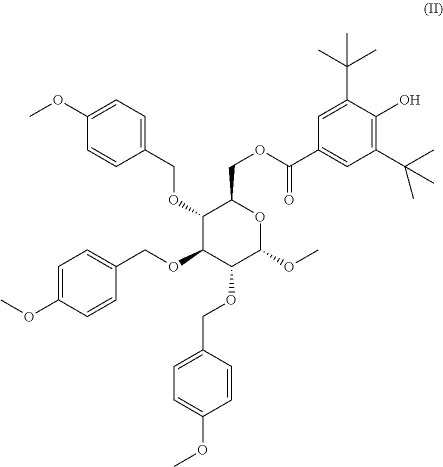

which, after deprotection, gives the esterified compound (I).

According to one advantageous embodiment of the process of the invention, the preparation of the acid chloride (III) is carried out starting from 3,5-di-tert-butyl-4-hydroxybenzoic acid of formula (IV):

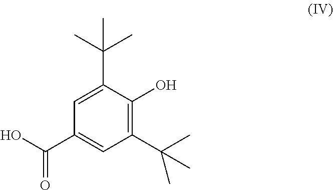

The synthesis of 3,5-di-tert-butyl-4-hydroxybenzoic acid of formula (IV), like that of the halides thereof, in particular the bromide and chloride, has been described in EP 0 269 981.

This acid (IV) has been proposed for preparing antiviral medicaments for treating diseases associated with an infection of an individual with viruses such as the herpesvirus or the AIDS virus.

According to another advantageous embodiment of the process of the invention, the preparation of the compound (V) comprises more particularly the following steps:
reacting 3,4,5-trihydroxy-6-methoxytetrahydropyran-2-ylmethanol of formula (VIII):

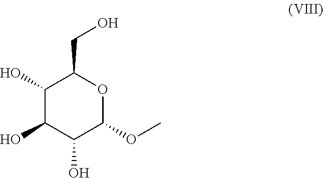

with p-anisaldehyde dimethyl acetal of formula (IX):

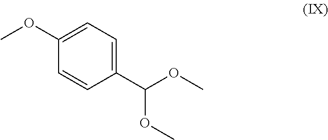

so as to obtain 6-methoxy-2-(4-methoxyphenyl)hexahydro-pyrano[3,2-D][1,3]dioxine-7,8-diol of formula (VII):

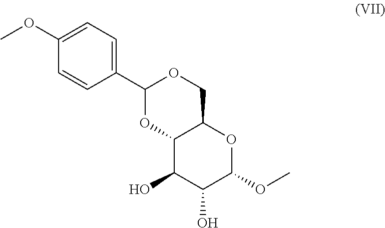

then reacting the resulting compound (VII) with p-methoxybenzyl chloride of formula (X):

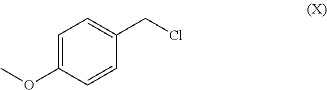

so as to obtain 6-methoxy-7,8-bis(4-methoxybenzyloxy)-2-(4-methoxyphenyl)hexahydropyrano[3,2-D][1,3]dioxine of formula (VI):

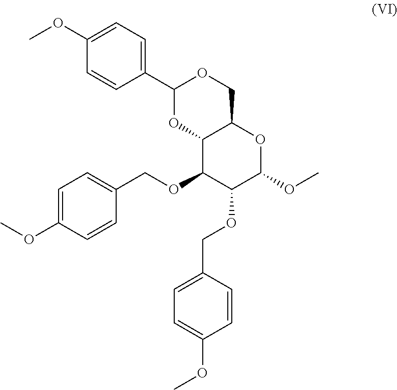

and, finally, reacting the resulting compound (VI) with sodium cyanoborohydride (NaBH₃CN) and chlorotrimethylsilane ((CH₃)₃SiCl) so as to obtain the compound of formula (V).

The compound (I) of the present invention has several advantages, in particular compared with the compound of patent FR 2 887 249 which has quite a close structure:
better water-solubility, which facilitates the production of a galenical preparation more suitable for a medicament,
a virucidal activity at lower concentrations.

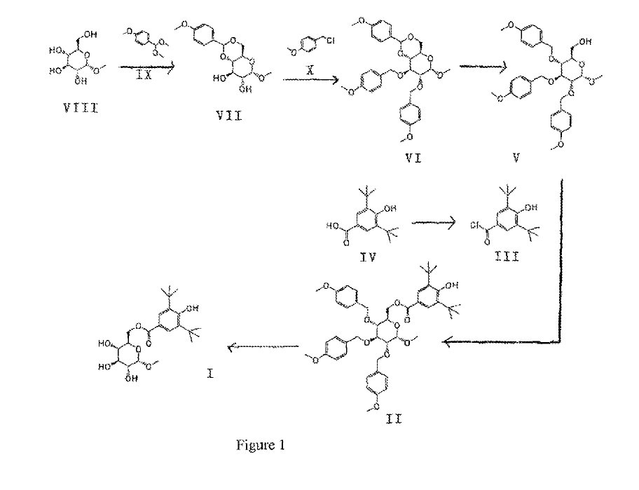

FIG. 1 represents the scheme for synthesis of the novel ester (I).

The following example illustrates the invention without limiting it in any way.

### EXAMPLE 1

### Synthesis of 3,4,5-Trihydroxy-6-Methoxy-Tetrahydropyran-2-Ylmethyl 3,5-Di-Tert-Butyl-4-Hydroxy-Benzoate of Formula (I) (See FIG. 1)

### 1) Synthesis of 3,5-Di-Tert-Butyl-4-Hydroxybenzoyl Chloride of Formula (III)

3,5-Di-tert-butyl-4-hydroxybenzoic acid (IV), purchased commercially, is activated in acid chloride form through the action of 5 equivalents of thionyl chloride (SOCl₂) at the reflux of toluene overnight at 110° C. The reaction medium is evaporated and the chlorinated compound (III) is obtained with a yield of 95%.

### 2) Synthesis of [6-Methoxy-3,4,5-Tris(4-Methoxy-Benzyloxy)Tetrahydropyran-2-yl]Methanol of Formula (V)

### a) Step 1: Synthesis of 6-methoxy-2-(4-methoxyphenyl)-hexahydropyrano[3,2-D][1,3]dioxine-7,8-diol of formula (VII)

The synthesis begins with the protection of the primary alcohol (VIII) (3,4,5-trihydroxy-6-methoxytetrahydro-pyran-2-ylmethanol) in the form of an acetal (VII) (6-methoxy-2-(4-methoxyphenyl)hexahydropyrano[3,2-D]-[1,3]dioxine-7,8-diol) through the action of p-anisaldehyde dimethyl acetal (III) in the presence of camphorsulfonic acid in DMF (dimethylformamide).

The reaction is carried out at ambient temperature under an industrial vacuum (pressure=40 mbar) (evaporation of the methanol form) for 2 hours, so that a significant advancement in the formation of the desired product is observed, such that only traces of the starting product remain.

After an aqueous treatment in a basic medium, the traces of starting product are eliminated by washing with cyclohexane; this makes it possible to isolate the compound (VII) in the form of a white solid with yields of between 60% and 80%.

### b) Step 2: synthesis of 6-methoxy-7,8-bis(4-methoxy-benzyloxy)-2-(4-methoxyphenyl)hexahydropyrano[3,2-D]-[1,3]dioxine of formula (VI)

The protection of the two free secondary alcohol functions of the compound (VII) is carried out through the action of sodium hydride and p-methoxybenzyl chloride (X) in a DMF/THF mixture (80/20).

The sodium hydride is added at 0° C., and then, after addition of the chlorinated derivative, heating at 90° C. maintained for 1 hour results in total formation of the protected product (VI).

Washing with cyclohexane results in a pure product being obtained in the form of a white solid and with a yield of 86%.

### c) Step 3: Synthesis of [6-methoxy-3,4,5-tris(4-methoxy-benzyloxy)tetrahydropyran-2-yl]methanol of formula (V)

The selective opening of the acetyl (VI) previously formed is carried out with sodium cyanoborohydride and chlorotrimethylsilane in the presence of 3 Å molecular sieve in acetonitrile. The reaction is carried out in 1 hour at −20° C., and, after basic treatment followed by silica gel purification, the opened product (V) is isolated in the form of a white solid with a yield of 60%.

### 3) Obtaining of 3,4,5-Trihydroxy-6-Methoxytetrahydro-Pyran-2-Ylmethyl 3,5-Di-Tert-Butyl-4-Hydroxybenzoate (I)

The coupling between the previously prepared acid chloride (III) and the opened compound (V) is carried out in the presence of 1.5 equivalents of triethylamine (Et₃N) in dichloromethane (CH₂Cl₂) at ambient temperature in 2 hours. The coupling product (II) (3,4,5-tris(4-methoxybenzyloxy)-6-methoxytetrahydro-pyran-2-ylmethyl 3,5-di-tert-butyl-4-hydroxybenzoate) is isolated by silica gel chromatography, in the form of a yellow oil with a yield of 70%.

The p-methoxybenzyl protections of the three secondary alcohol positions of the compound (II) are cleaved with trifluoroacetic acid (12 equivalents) in dichloro-methane (CH₂Cl₂).

After an overnight period at ambient temperature, all of the compound (II) is converted to free compound (I). It is isolated by being rapidly passed over silica, to give a white solid with a yield of 60%.

10.5 g of 3,4,5-trihydroxy-6-methoxytetrahydropyran-2-ylmethyl 3,5-di-tert-butyl-4-hydroxybenzoate are thus obtained with an HPLC purity of 99.3% and an overall synthesis yield of 15%.

1. The compound 3,4,5-trihydroxy-6-methoxytetrahydropyran-2-ylmethyl 3,5-di-tert-butyl-4-hydroxybenzoate defined by formula (I) below:

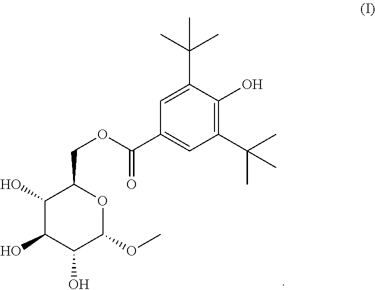

2. The compound as claimed in claim 1, characterized in that it is in the form of a salt or of an acid of said compound (I).

3. A composition, characterized in that it comprises at least one compound as claimed in claim 1, in combination with a biologically active compound.

4. A pharmaceutical composition, characterized in that it contains a therapeutically effective amount of at least one compound or of at least one composition as claimed in claim 1.

5. The pharmaceutical composition as claimed in claim 4, characterized in that it is in a form suitable for oral, injectable or parenteral administration.

6. The use of a compound or of a composition as claimed in claim 1, for preparing a medicament for treating and/or preventing enveloped virus infections.

7. A process for preparing a compound of formula (I) as claimed in claim 1, characterized in that it comprises reacting 3,5-di-tert-butyl-4-hydroxybenzoyl chloride of formula (III): 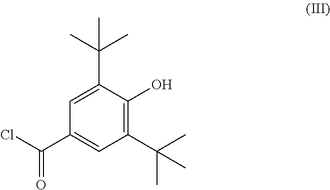
with [6-methoxy-3,4,5-tris(4-methoxybenzyloxy)tetrahydropyran-2-yl]methanol of formula (V): 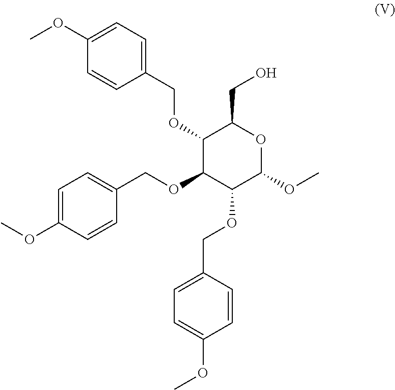
so as to obtain the esterified compound “3,4,5-tris(4-methoxybenzyloxy)-6-methoxytetrahydropyran-2-ylmethyl 3,5-di-tert-butyl-4-hydroxybenzoate” of formula (II): 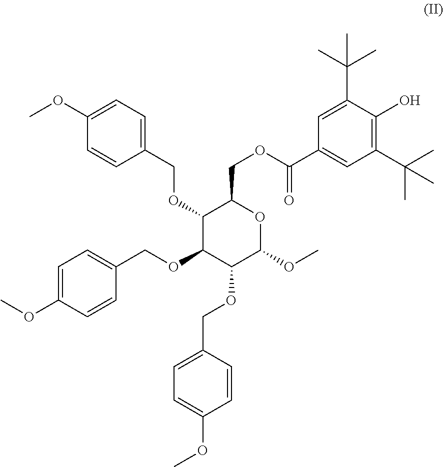
which, after deprotection, gives the esterified compound (I).

8. The preparation process as claimed in claim 7, characterized in that the preparation of the 3,5-di-tert-butyl-4-hydroxybenzyl chloride of formula (III) is carried out starting from 3,5-di-tert-butyl-4-hydroxybenzoic acid of formula (IV):

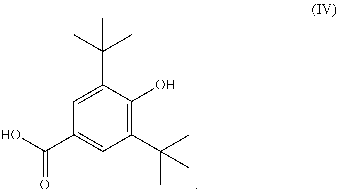

9. The preparation process as claimed in claim 7, characterized in that the preparation of the [6-methoxy-3,4,5-tris(4-methoxybenzyloxy)tetrahydropyran-2-yl]methanol of formula (V) comprises the following steps:
reacting 3,4,5-trihydroxy-6-methoxytetrahydropyran-2-ylmethanol of formula (VIII): 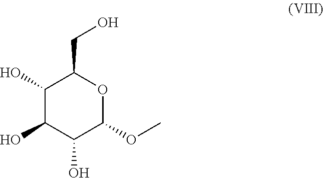
with p-anisaldehyde dimethyl acetal of formula (IX): 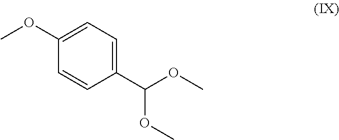
so as to obtain 6-methoxy-2-(4-methoxyphenyl)hexahydro-pyrano[3,2-D][1,3]dioxine-7,8-diol of formula (VII): 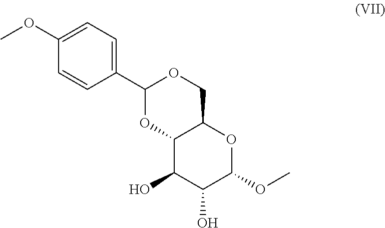
then reacting the resulting compound (VII) with p-methoxybenzyl chloride of formula (X): 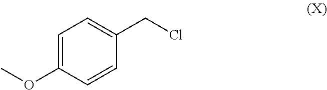
so as to obtain 6-methoxy-7,8-bis(4-methoxybenzyloxy)-2-(4-methoxyphenyl)hexahydropyrano[3,2-D][1,3]dioxine of formula (VI): 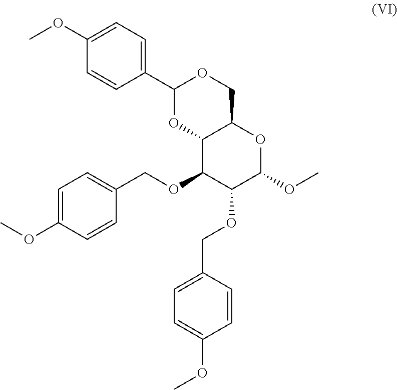
and, finally, reacting the resulting compound (VI) with sodium cyanoborohydride (NaBH₃CN) and chlorotrimethylsilane ((CH₃)₃SiCl) so as to obtain the compound of formula (V).
